(19) United States
(12) Patent Application Publication
(10) Pub. No.: US 2020/0040296 A1
(43) Pub. Date: Feb. 6, 2020

(54) Decellularized Tissue as a Microcarrier for Cell Culture and Expansion

(71) Applicant: UNIVERSITY OF SOUTH CAROLINA, Columbia, SC (US)

(72) Inventor: Esmaiel JABBARI, Bethesda, MD (US)

(21) Appl. No.: 16/511,431

(22) Filed: Jul. 15, 2019

Related U.S. Application Data
(60) Provisional application No. 62/714,793, filed on Aug. 6, 2018.

(51) Int. Cl. C12M 1/34; C12N 5/00

(52) U.S. Cl. CPC C12M 41/32; C12N 5/0018; C12N 5/0062; C12N 2531/00; C12N 2533/90; C12N 2537/10; C12N 2535/10; C12N 5/0068

(57) ABSTRACT

A microcarrier for cell culture and expansion is provided. The microcarrier includes decellularized mammalian tissue. Further, the microcarrier has an average particle size ranging from about 10 micrometers to about 600 micrometers. A method of forming a decellularized mammalian tissue microcarrier for cell culture and expansion is also provided, along with a method for treating a mammalian tissue defect via a decellularized mammalian tissue microcarrier on which cells from the same tissue type as the decellularized mammalian tissue are expanded.

### CROSS-REFERENCE TO RELATED APPLICATION

The present application claims priority to U.S. Provisional Application Ser. No. 62/714,793, filed on Aug. 6, 2018, the disclosure of which is incorporated by reference herein.

### BACKGROUND

The cultivation and expansion of mammalian cells, including human and other animal cells, on two-dimensional (2D) tissue culture plates or three-dimensional (3D) suspended microbead/microcarrier (MC) tissue culture systems are not benign processes. To expand cells outside (in vitro) the natural tissue environment, first, the cells must be released from the natural tissue by treating the tissue with enzymes to degrade the extracellular matrix (ECM) and detach and isolate the cells from the ECM and other cells. Next, the cells are seeded on 2D adherent culture plates or 3D adherent MCs at low density. The cells attach to the surface of the tissue culture plate or the MCs and begin to proliferate and expand. After reaching confluency, the seeded cells are detached from the surface of culture plates or MC using an enzyme like trypsin. After removing the enzyme and, if utilized MCs, from the resulting suspension containing the detached cells, the cells are split and re-seeded on new culture plates or new MCs for further expansion. The in vitro cultured cells (on flasks, plates, or MCs) are expanded in an environment that is considerably different from that in the natural tissue.

Specifically, cells in vivo reside within an interconnected 3D structure, whereas cells on culture plates or MCs are highly polarized, as part of the cell is firmly attached to the rigid surface of the plate or MCs while the rest of the cell is in contact with the culture medium. This polarization leads to cellular shock and stress which, in turn, activates the expression of reactive oxygen species (ROS) and other toxic species. The release of ROS and toxic species cause DNA damage, cell mutation, and tumorigenesis. Further, the cell shape on culture plates or MCs is considerably different from the in vivo shape within the tissue, which dramatically affects cell phenotype, lineage determination, and fate. As a result, there is undeniable uncertainty regarding the fate of plated or MC-cultured cells with respect to transplantation and clinical applications. In addition, the lack of tissue-specific extracellular matrix (ECM) in plated/MC cell cultures affects cell phenotype and fate.

Further, an important consideration in the development of regenerative therapies is the implementation of cell biomanufacturing methods that are sufficient to produce large quantities of cells from an initial small number of patient-derived cells. For instance, regenerative therapies often require tens of billions of cells from an initial batch of approximately tens of millions of cells. Such large expansion of cells using 2D tissue culture flasks by thousands of folds is not economically feasible on a commercial scale. However, to overcome this limitation, 3D microcarrier-based (MC) suspension cultures as a platform for cell expansion have been developed. One of the advantages of the MC platform is the high surface to volume ratio compared to 2D culture flasks, where the microcarriers can be solid or porous. Porous MCs provide much higher surface area for cell expansion per unit volume. The commercially available solid MCs include among others Cytodex™ made from dextran and SoloHill™ made from polystyrene. The commercially available porous MCs include, among others, CultiSpher™ made from porcine gelatin, Cytopore™ made from cylcodextran, and Cytoline™ based on macroporous polyethylene. Although such MC platforms provide much greater surface area and a more biologically relevant 3D geometry for cell expansion compared to 2D culture vessels such as flasks or plates, there are limitations associated with MCs. Specifically, as the commercially available MCs are either non-biodegradable, synthetic, or all-purpose type of biomolecules, the commercially available MCs have to be separated from the cells being expanded on the MCs in order to use the cells for therapeutic applications, and trypsin and other enzymes added to repetitively detach and separate cells from the MCs for cell expansion remove adhesion proteins from the cell surface, which negatively affect cell function and fate. Further, the all-purpose nature of natural matrices like gelatin affects differentiation and maturation of expanded cells to a specified lineage and phenotype.

As such, a need exists for a cell culture system and method for the expansion of mammalian cells that more closely mimics the natural in vivo environment in which micronized decellularized tissue of origin of the cells is used for cell culture. Moreover, an ideal microcarrier for cell expansion and transplantation should not need to be separated from the cells and should be an integral part of the final cellular construct for transplantation. Further, a need exists for a viable mechanism for passaging the cells via attachment and detachment of the cells from MCs for expansion where the cells do not need to be removed from the MCs prior to transplantation. A cell culture system and method that also provides a tissue-specific in vivo-mimetic matrix to enhance cell phenotype and function would also be beneficial.

### SUMMARY OF THE INVENTION

In one particular embodiment of the present invention, a microcarrier for cell culture and expansion is provided. The microcarrier includes decellularized mammalian tissue. Further, the microcarrier has an average particle size ranging from about 10 micrometers to about 600 micrometers.

In one embodiment, the decellularized mammalian tissue can originate from a human donor, a specific human patient, or a non-human mammal.

In another embodiment, the decellularized mammalian tissue can originate from embryonic tissue, neonatal tissue, natal tissue, juvenile tissue, or adult tissue.

In one more embodiment, the decellularized mammalian tissue can originate from articular cartilage tissue, bone tissue, heart tissue, liver tissue, skin tissue, or gall bladder tissue.

In another embodiment, the microcarrier can be compatible with cells harvested from mammalian tissue of a same type as the decellularized mammalian tissue.

In yet another embodiment, the decellularized mammalian tissue can be micronized.

In still another embodiment, the decellularized mammalian tissue can be digested and functionalized. Further, the decellularized mammalian tissue can be cross-linked.

In an additional embodiment, the microcarrier can have a honeycomb microstructure.

In one more embodiment, the microcarrier can include pores having an average pore size ranging from about 2.5 micrometers to about 20 micrometers.

In another particular embodiment of the present invention, an injectable or implantable tissue regeneration product is provided. The implantable tissue regeneration product includes a microcarrier for cell culture and expansion, where the microcarrier includes decellularized mammalian tissue and has an average particle size ranging from about 10 micrometers to about 600 micrometers; mammalian cells harvested from mammalian tissue of a same type as the decellularized mammalian tissue; one or more growth factors; and a gel precursor or a pre-formed scaffold.

In one more embodiment, the decellularized mammalian tissue can originate from a human donor, a specific human patient, or a non-human mammal.

In another embodiment, the decellularized mammalian tissue can originate from embryonic tissue, neonatal tissue, natal tissue, juvenile tissue, or adult tissue.

In still another embodiment, the decellularized mammalian tissue can originate from articular cartilage tissue, bone tissue, heart tissue, liver tissue, skin tissue, or gall bladder tissue.

In one embodiment, the decellularized mammalian tissue can be digested and functionalized, further wherein the injectable or implantable tissue regeneration product can include a cross-linking agent.

In yet another particular embodiment of the present invention, a method of forming a decellularized mammalian tissue microcarrier for cell culture and expansion is provided. The method includes obtaining a mammalian tissue sample; decellularizing the mammalian tissue sample; and micronizing the mammalian tissue sample to form the decellularized mammalian tissue microcarrier, where the decellularized mammalian tissue microcarrier has an average particle size ranging from about 10 micrometers to about 600 micrometers.

In one embodiment, the method further includes digesting the decellularized mammalian tissue sample; functionalizing the decellularized mammalian tissue sample; and crosslinking the decellularized mammalian tissue sample.

In another embodiment, the mammalian tissue sample can originate from a human donor, a specific human patient, or a non-human mammal.

In one more embodiment, the mammalian tissue sample can originate from embryonic tissue, neonatal tissue, natal tissue, juvenile tissue, or adult tissue.

Moreover, the mammalian tissue sample can originate from articular cartilage tissue, bone tissue, heart tissue, liver tissue, skin tissue, or gall bladder tissue.

In yet another embodiment, the decellularized mammalian tissue microcarrier can be compatible with cells harvested from mammalian tissue of a same type as the mammalian tissue sample.

In still another embodiment, the decellularized mammalian tissue microcarrier can have a honeycomb microstructure.

In an additional embodiment, the decellularized mammalian tissue microcarrier can include pores having an average pore size ranging from about 2.5 micrometers to about 20 micrometers.

In one more embodiment, the decellularized mammalian tissue microcarrier can be freeze-dried.

In still another particular embodiment of the present invention, a method of treating a mammalian tissue defect is provided. The method includes obtaining a decellularized mammalian tissue microcarrier, wherein the decellularized mammalian tissue microcarrier has an average particle size ranging from about 10 micrometers to about 600 micrometers; expanding cells harvested from a mammalian tissue sample of a same type as the decellularized mammalian tissue microcarrier on the decellularized mammalian tissue microcarrier inside a cell culture vessel; and injecting or implanting the decellularized mammalian tissue microcarrier with the expanded cells attached thereto into the mammalian tissue defect.

In one embodiment, the decellularized mammalian tissue microcarrier with the expanded cells attached thereto can be injected into the tissue defect in conjunction with one or more growth factors and a gel precursor, further wherein a cross-linking agent can be injected into the tissue defect.

In another embodiment, the decellularized mammalian tissue microcarrier with the expanded cells attached thereto can be loaded into a pre-formed scaffold containing one or more growth factors, wherein the pre-formed scaffold containing the one or more growth factors and the decellularized mammalian tissue microcarrier with the expanded cells attached thereto is implanted into the tissue defect.

In one embodiment, the decellularized mammalian tissue microcarrier can originate from a human donor, a specific human patient, or a non-human mammal.

In another embodiment, the decellularized mammalian tissue microcarrier can originate from embryonic tissue, neonatal tissue, natal tissue, juvenile tissue, or adult tissue.

In still another embodiment, the decellularized mammalian tissue microcarrier can originate from articular cartilage tissue, bone tissue, heart tissue, liver tissue, skin tissue, or gall bladder tissue.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures, in which:

FIG. 1 illustrates a method of forming a cell culture system in the form of decellularized tissue as a microcarrier according to one embodiment of the present invention;

FIG. 2 illustrates a method of forming a cell culture system in the form of digested decellularized tissue as a microcarrier according to one embodiment of the present invention;

FIG. 3 illustrates a cell expansion method using decellularized tissue as a microcarrier according to one embodiment of the present invention;

FIG. 4 illustrates a cell expansion method using digested decellularized tissue as a microcarrier according to one embodiment of the present invention; and

FIG. 5 illustrates a method for in situ regeneration of tissue according to one embodiment of the present invention.

Repeat use of reference characters in the present specification and drawings is intended to represent the same or analogous features or elements of the present invention.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only and is not intended as limiting the broader aspects of the present invention, which broader aspects are embodied exemplary constructions.

Generally speaking, the present invention is directed to a cell culture system and method for the expansion of mammalian cells in which micronized, decellularized mammalian tissue from which the mammalian cells originate is used as the substrate on which the cells are seeded for expansion. For instance, the present invention contemplates the use of three-dimensional micronized decellularized tissue or three-dimensional micronized digested decellularized tissue as a platform to culture and expand cells corresponding to that specific tissue. The decellularized tissue can come from a mammal, a human cadaver, the patient to be treated, or from any other suitable tissue source. For instance, in one embodiment, the decellularized tissue can originate from a human donor, a specific human patient, or a non-human mammal. In another embodiment, the decellularized tissue can originate from embryonic tissue, neonatal tissue, natal tissue, juvenile tissue, or adult tissue. In one more embodiment, the decellularized tissue can originate from articular cartilage tissue, bone tissue, heart tissue, liver tissue, skin tissue, or gall bladder tissue.

Meanwhile, the cells can be harvested from the patient to be treated. In one embodiment, the mammalian tissue can be articular cartilage, where the decellularized articular cartilage is referred to as DAC and the digested decellularized articular cartilage is referred to as DDAC. However, the approach described in the present invention can be extended to other mammalian tissues such as bone, heart, liver, skin, gall bladder, etc.

As a result of the methods and systems contemplated by the present invention, cells ultimately used as a component of an in situ tissue regeneration product are not stressed during culture and expansion, and thus, do not experience a shock because the cells are in contact with a decellularized or digested and decellularized matrix that is almost identical to the cells' native tissue environment. Further, during culture and expansion, the cells are detached from the decellularized or digested and decellularized microcarrier using the same enzymes used to dislodge and release therapeutic cells from the native tissue origin, which can enhance the compatibility of the cells and the microcarrier. Moreover, the expanded cells on the microcarrier can be transplanted directly into a tissue defect or a compromised or otherwise injured area of tissue without the need to remove the microcarrier because the microcarrier is enzymatically-degradable and based on the tissue from which the cells attached to the microcarrier have originated. In addition, the cells are expanded within the honeycomb microstructure of a decellularized or digested and decellularized microcarrier. Therefore, present invention contemplates a microscale cell expansion platform for commercial and regenerative medicine applications. Additionally, the cells attached to the microcarriers of the present invention experience a balance of cell-cell and cell-matrix interaction similar to what the cells experience in vivo based on the similarity of the microcarrier to the cells' native environment.

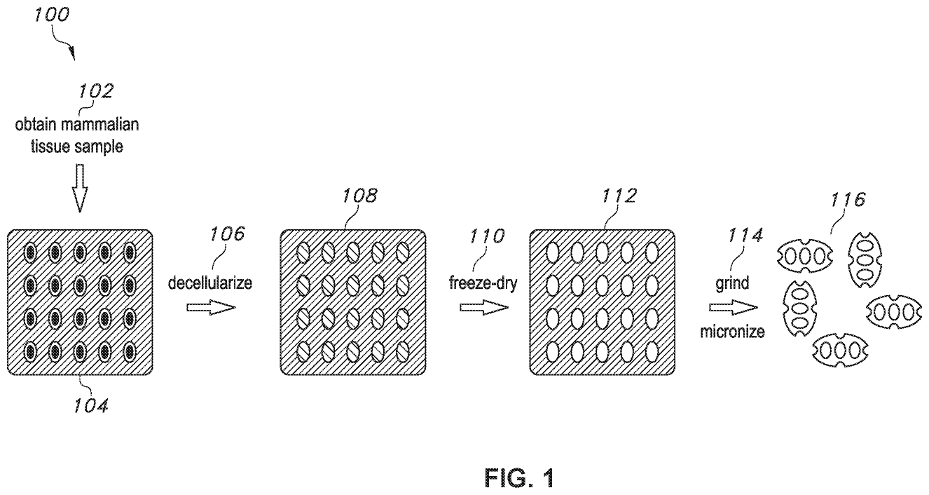

Specifically, FIG. 1 illustrates a method 100 of forming a cell culture system in the form of decellularized tissue as a microcarrier according to one embodiment of the present invention. First, in step 102, a mammalian tissue sample 104 is obtained from any suitable source (e.g., an animal, a human cadaver, the patient to be treated, etc.). If necessary, the mammalian tissue sample 104 can be thawed, after which the mammalian tissue sample 104 is dissected into small pieces with a scalpel to generate millimeter scale fragments of tissue, such as tissue having length, width, and height dimensions each ranging from about 0.5 millimeters (mm) to about 4 mm, such as from about 1 mm to about 3.5 mm, such as from about 1.5 mm to about 3 mm.

Then, in step 106, the mammalian tissue sample 104, now in millimeter scale fragments of tissue, is decellularized, leaving a decellularized mammalian tissue sample 108. For instance, the millimeter scale mammalian tissue fragments can be incubated in a cell lysis buffer, such as a buffer containing 10 mM (hydroxymethyl)aminomethane hydrochloride (Tris-HCl) and 1% polyethylene glycol p-(1,1,3,3-tetramethylbutyl)-phenyl ether (Triton X-100), for a time frame ranging from about 12 hours to about 36 hours, such as from about 18 hours to about 30 hours, such as about 24 hours under ambient conditions (e.g., at about 23° C. and about 70% relative humidity) and stirred. Next, the resulting suspension can be sonicated at a frequency ranging from about 40 kilohertz (kHz) to about 70 kHz, such as from about 45 kHz to about 65 kHz, such as from about 50 kHz to about 60 kHz for a time frame ranging from about 30 minutes to about 4 hours, such as from about 60 minutes to about 3 hours, such as from about 90 minutes to about 2 hours under ambient conditions. The supernatant can then be removed and the tissue fragments can be washed, such as with phosphate buffer saline (PBS). Then, the tissue fragments can be incubated in a nuclease solution containing 1 U/mL deoxyribonuclease and 1 U/mL ribonuclease in PBS for a time frame ranging from about 24 hours to about 120 hours, such as from about 36 hours to about 108 hours, such as from about 48 hours to about 96 hours, at 37° C. under stirring to remove all nucleic acids (DNA and RNA). Next, the tissue fragments can be washed thoroughly with PBS leaving a decellularized mammalian tissue sample 108. Then, in step 110, the decellularized mammalian tissue sample 108 is freeze-dried, leaving a freeze-dried decellularized mammalian tissue sample 112, which can be stored at −20° C. until needed for further use.

Thereafter, in step 114, the freeze-dried decellularized mammalian tissue sample 112 is ground or micronized to form a decellularized mammalian tissue microcarrier 116 that can have a honeycomb microstructure. To micronize the freeze-dried decellularized mammalian tissue sample 112, the freeze-dried decellularized mammalian tissue sample 112 can be placed in a mortar filled with liquid nitrogen, and the freeze-dried decellularized mammalian tissue sample 112 can be crushed and grinded with a pestle. However, it is to be understood that other micronization methods known in the art can also be used for micronization as long as the method does not require heating the tissue fragments above the physiological temperature of 37° C. Next, the crushed tissue sample is passed through a sieve to reduce the particle size to less than about 300 micrometers (μm). The average particle size of the decellularized mammalian tissue microcarrier 116 after micronization can range from about 10 μm to about 600 such as from about 25 μm to 550 μm, such as from about 50 μm to about 500 μm by changing the parameters of micronization process. In one particular embodiment, the average particle size of the decellularized mammalian tissue microcarrier 116 after micronization can range from about 150 μm to about 250 μm. The resulting decellularized mammalian tissue microcarriers 116 can have random geometries and may not necessarily be spherical. The decellularized mammalian tissue microcarriers 116 can be porous with a honeycomb microstructure with average pore size ranging from about 2.5 μm to about 20 such as from about 5 μm to about 15 such as from about 7.5 μm to about 12.5 μm.

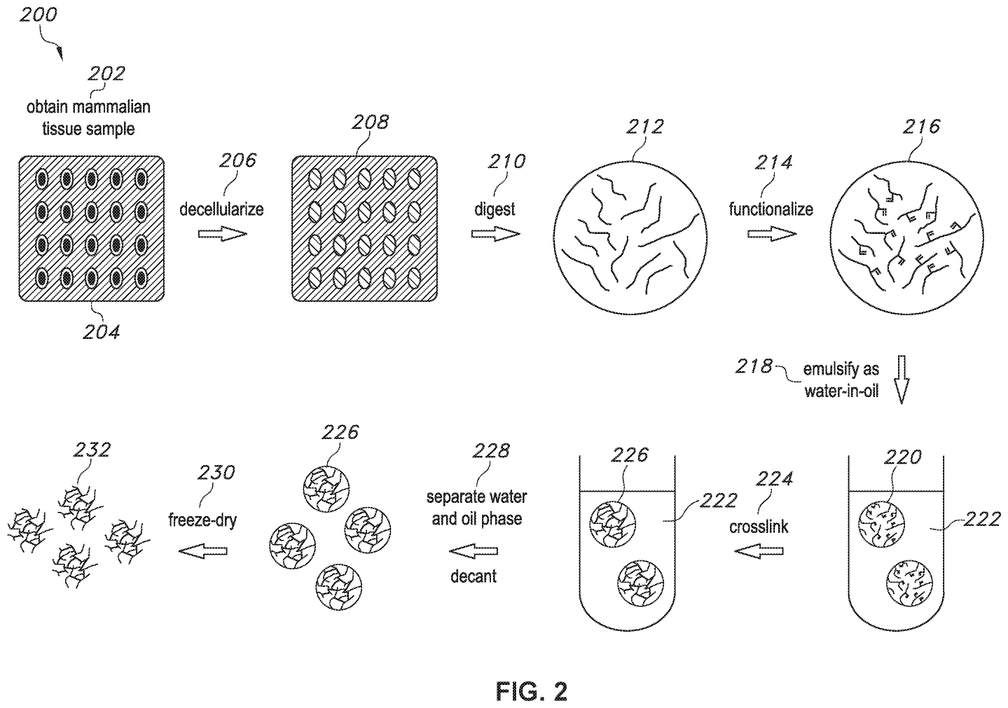

Turning now to FIG. 2, a method 200 of forming a cell culture system in the form of digested decellularized tissue as a microcarrier according to one embodiment of the present invention is illustrated. First, in step 202, a mammalian tissue sample 204 is obtained from any suitable source (e.g., an animal, a human cadaver, the patient to be treated, or any other source such as embryonic tissue, fetal tissue, neonatal tissue, natal tissue, juvenile tissue, or adult tissue, etc.). If necessary, the mammalian tissue sample 204 can be thawed, after which the mammalian tissue sample 204 is dissected into small pieces with a scalpel to generate millimeter scale fragments of tissue, such as tissue having length, width, and height dimensions each ranging from about 0.5 millimeters (mm) to about 4 mm, such as from about 1 mm to about 3.5 mm, such as from about 1.5 mm to about 3 mm.

Then, in step 206, the mammalian tissue sample 204, now in millimeter scale fragments of tissue, is decellularized, leaving a decellularized mammalian tissue sample 208. For instance, the millimeter scale mammalian tissue fragments can be incubated in a cell lysis buffer, such as a buffer containing 10 mM (hydroxymethyl)aminomethane hydrochloride (Tris-HCl) and 1% polyethylene glycol p-(1,1,3,3-tetramethylbutyl)-phenyl ether (Triton X-100), for a time frame ranging from about 12 hours to about 36 hours, such as from about 18 hours to about 30 hours, such as about 24 hours under ambient conditions (e.g., at about 23° C. and about 70% relative humidity) and stirred. Next, the resulting suspension can be sonicated at a frequency ranging from about 40 kilohertz (kHz) to about 70 kHz, such as from about 45 kHz to about 65 kHz, such as from about 50 kHz to about 60 kHz for a time frame ranging from about 30 minutes to about 4 hours, such as from about 60 minutes to about 3 hours, such as from about 90 minutes to about 2 hours under ambient conditions. The supernatant can then be removed and the tissue fragments can be washed, such as with phosphate buffer saline (PBS). Then, the tissue fragments can be incubated in a nuclease solution containing 1 U/mL deoxyribonuclease and 1 U/mL ribonuclease in PBS for a time frame ranging from about 24 hours to about 120 hours, such as from about 36 hours to about 108 hours, such as from about 48 hours to about 96 hours, at 37° C. under stirring to remove all nucleic acids (DNA and RNA). Next, the tissue fragments can be washed thoroughly with PBS leaving a decellularized mammalian tissue sample 208.

Thereafter, in step 210, the decellularized mammalian tissue sample 208 is digested with a digestive enzyme such as pepsin or any other suitable digestive enzyme in a 0.01 M hydrochloric acid solution at 37° C. using well-established protocols until a clear suspension of digested decellularized mammalian tissue 212 is obtained. Tissue digestion is complete when the suspension turns from milky or translucent color to a clear suspension. Next, the digestion solution pH is raised to 9.0 using 1 M NaOH to inactivate the digestive enzyme. After enzyme deactivation, the pH is adjusted to 7.5 using 1 M HCl.

Next, in step 214, the digested decellularized mammalian tissue 212 is functionalized. For instance, to functionalize the digested decellularized mammalian tissue 212 to obtain functionalized digested decellularized mammalian tissue 216, a functionalization agent such as methacrylic anhydride can be added dropwise to the solution (using from about 1 mL to about 5 mL, such as from about 1.5 mL to about 4 mL, such as about 2 mL to about 3 mL, such as about 2.5 mL of functionalization agent per gram of digested decellularized tissue 212) under rigorous stirring to functionalize the digested decellularized mammalian tissue 212 using well-established methods in the art. The reaction can be allowed to proceed for a time from ranging from about 15 minutes to about 120 minutes, such as from about 30 minutes to about 105 minutes, such as from about 45 minutes to about 90 minutes. After reaction, the resulting solution can be dialyzed against distilled water at ambient condition to remove the unreacted functionalization agent (e.g., methacrylic anhydride). The resulting functionalized digested decellularized macromere solution can then be freeze-dried or lyophilized to obtain a white foam, hereafter referred to as the functionalized digested decellularized mammalian tissue 216, and stored at a temperature of about −20° C.

Next, to micronize the functionalized digested decellularized mammalian tissue 216, in step 218, a water phase 220 of the functionalized digested decellularized mammalian tissue 216 can be prepared by dissolving the functionalized digested decellularized mammalian tissue 216 in an aqueous solution containing a water soluble emulsifier, such as polyvinyl alcohol, polyethylene glycol based emulsifiers, biodegradable poly(ethylene oxide-co-lactide-fumarate) (PLEOF) emulsifiers, or any other water soluble emulsifiers as known in the art. Chemical agents such as a crosslinking initiator, crosslinking co-initiator, a crosslinking agent, or a combination thereof can also be added to the water phase 220 for crosslinking the functionalized digested decellularized mammalian tissue 216 in the water phase 220 if desired, as shown in step 224. Next, as shown in step 218, the water phase 220 can be emulsified in an oil phase 222 that can include an edible oil such as palm oil or any other edible oil known in the art. After emulsification, the functionalized digested decellularized mammalian tissue 216 in the water phase 220 in the form of droplets can be allowed to gel to stabilize the droplets in oil and form microbeads (MBs) as microcarriers or functionalized digested decellularized mammalian tissue microgels 226 suspended in the oil phase 222. Next, in step 228, the MBs or microgels 226 can be separated from the oil phase 222 by centrifugation or other methods known in the art and decanted or washed thoroughly with water to remove unreacted compounds, emulsifier, crosslinking initiator and co-initiator, and crosslinking agent. Next, in step 230, the MBs or microgels 226 are suspended in water and stored frozen at about −20° C. This approach results in porous spherical microbeads 232 as microcarriers with an average particle size varying from about 10 μm to about 600 μm, such as from about 25 μm to 550 μm, such as from about 50 μm to about 500 μm.

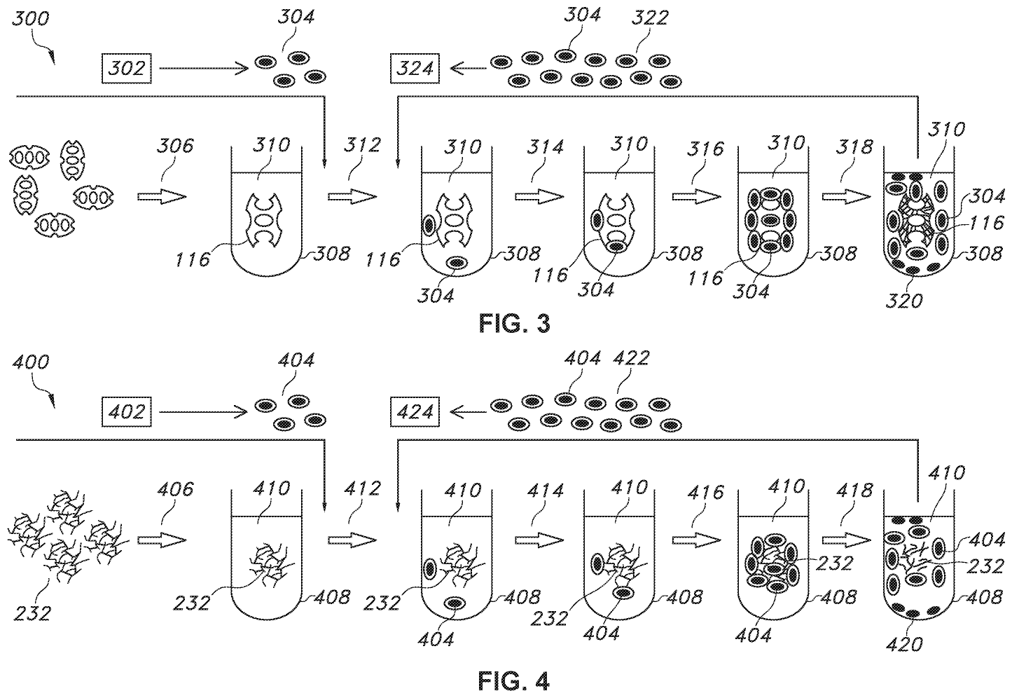

Regardless of whether decellularized mammalian tissue microcarriers 116 are formed as set forth in FIG. 1 or whether functionalized digested decellularized mammalian tissue microcarriers 232 are formed as set forth in FIG. 2, the resulting microcarriers can be used for cell expansion methods 300 and 400 as shown in FIGS. 3 and 4, respectively.

Turning first to FIG. 3 a cell expansion method 300 using decellularized mammalian tissue microcarriers 116 is illustrated. First, in step 302, mammalian tissue from a patient can be harvested and treated with matrix-degrading enzymes to release the cells 304 present in the mammalian tissue. Alternatively, the cells 304 can be harvested from the patient's bone marrow or fat tissue via a biopsy procedure using well-established protocols. The harvested cells 304 can then be washed, sorted, and characterized, and the desired cells 304 can be selected for expansion.

Meanwhile, after suspending the decellularized mammalian tissue microcarriers 116 in cell culture medium 310 in a stirred-tank cell bioreactor or other suitable culture vessel 308 in step 306, the desired cells 304 can be added to the bioreactor 308 in step 312. Desirably, a non-adherent tissue culture bioreactor should be used to block cell attachment to the reactor surface and promote cell attachment to the decellularized mammalian tissue microcarriers 116.

Next, in step 314, the cells 304/cell culture medium 310 suspension can be cultured in a tissue culture incubator (37° C., 100% humidity, 5% CO₂) for a time period ranging from about 6 hours to about 96 hours, such as from about 12 hours to about 72 hours, such as from about 24 hours to about 48 hours, to allow for sufficient attachment of the cells 304 to the decellularized mammalian tissue microcarriers 116.

Then, the cells 304/cell culture medium 310 suspension can be centrifuged, the supernatant containing the unattached cells can be removed, and the pellet containing the cells 304 attached to the decellularized mammalian tissue microcarriers 116 can be re-suspended in the desired cell culture medium 310 for cell growth and expansion in step 316. During expansion in step 316, the cell culture medium 310 can be replaced with fresh medium every few days by centrifugation and re-suspension of the decellularized mammalian tissue microcarriers 116 laden with cells 304 to replenish the nutrients in the cell culture medium 310. Furthermore, in step 316, the cells 304 can proliferate and expand on the decellularized mammalian tissue microcarriers 116 until the cells 304 reach the desired confluency.

Next, once the cells 304 grow and reach confluency on the decellularized mammalian tissue microcarriers 116, the cell culture medium 310 can be replaced with basal medium containing matrix-degrading enzymes 320 in step 318, which are the same enzymes used to release the cells 304 from the mammalian tissue in step 302. Then, after digestion of the decellularized mammalian tissue microcarriers 116, the resulting suspension can be centrifuged, the basal medium can be removed, and the pellet of expanded cells 322 can be re-suspended and added to a new suspension of decellularized mammalian tissue microcarriers 116 in step 324 to repeat the process set forth above. This process can be repeated many times until the desired fold increase in cell number is reached, where it should be understood that each batch of decellularized mammalian tissue microcarriers 116 has the potential to increase the cell number by 1000 fold. Therefore, expansion with one decellularized mammalian tissue microcarrier 116 change can potentially lead to a 1 million-fold increase in cell number.

On the other hand, FIG. 4 illustrates a cell expansion method 400 using functionalized digested decellularized tissue microcarriers 232. First, in step 402, mammalian tissue from a patient can be harvested and treated with matrix-degrading enzymes to release the cells 404 present in the mammalian tissue. Alternatively, the cells 404 can be harvested from the patient's bone marrow or fat tissue via a biopsy procedure using well-established protocols. The harvested cells 404 can then be washed, sorted, and characterized, and the desired cells 404 can be selected for expansion.

Meanwhile, after suspending functionalized digested decellularized tissue microcarriers 232 in cell culture medium 410 in a stirred-tank cell bioreactor or other suitable culture vessel 408 in step 406, the desired cells 404 can be added to the bioreactor 308 in step 412. Desirably, a non-adherent tissue culture bioreactor should be used to block cell attachment to the reactor surface and promote cell attachment to the functionalized digested decellularized tissue microcarriers 232.

Next, in step 414, the cells 404/cell culture medium 410 suspension can be cultured in a tissue culture incubator (37° C., 100% humidity, 5% CO₂) for a time period ranging from about 6 hours to about 96 hours, such as from about 12 hours to about 72 hours, such as from about 24 hours to about 48 hours, to allow for sufficient attachment of the cells 404 to the functionalized digested decellularized tissue microcarriers 232.

Then, the cells 304/cell culture medium 310 suspension can be centrifuged, the supernatant containing the unattached cells can be removed, and the pellet containing the cells 404 attached to the functionalized digested decellularized tissue microcarriers 232 can be re-suspended in the desired cell culture medium 410 for cell growth and expansion in step 416. During expansion in step 416, the cell culture medium 410 can be replaced with fresh medium every few days by centrifugation and re-suspension of the functionalized digested decellularized tissue microcarriers 232 laden with cells 404 to replenish the nutrients in the cell culture medium 410. Furthermore, in step 416, the cells 404 can proliferate and expand on the functionalized digested decellularized tissue microcarriers 232 until the cells 404 reach the desired confluency.

Next, once the cells 404 grow and reach confluency on the functionalized digested decellularized tissue microcarriers 232, the cell culture medium 410 can be replaced with basal medium containing matrix-degrading enzymes 420 in step 418, which are the same enzymes used to release the cells 404 from the mammalian tissue in step 402. Then, after digestion of the functionalized digested decellularized tissue microcarriers 232, the resulting suspension can be centrifuged, the basal medium can be removed, and the pellet of expanded cells 422 can be re-suspended and added to a new suspension of functionalized digested decellularized tissue microcarriers 232 in step 424 to repeat the process set forth above. This process can be repeated many times until the desired fold increase in cell number is reached, where it should be understood that each batch of functionalized digested decellularized tissue microcarriers 232 has the potential to increase the cell number by 1000 fold. Therefore, expansion with one functionalized digested decellularized tissue microcarrier 232 change can potentially lead to a 1 million-fold increase in cell number.

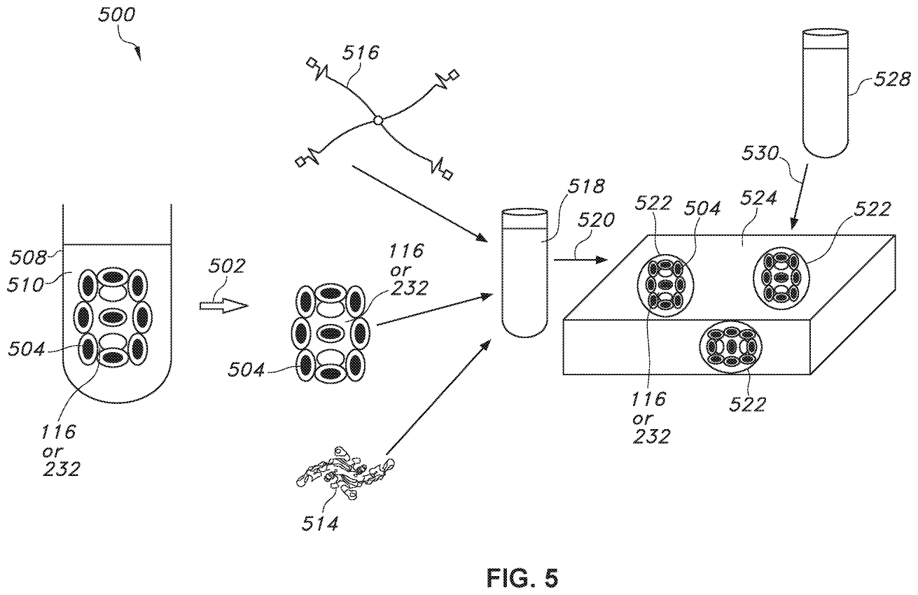

Lastly, FIG. 5 illustrates a method 500 for in situ regeneration of tissue according to one embodiment of the present invention. Specifically, cells 504 expanded on decellularized mammalian tissue microcarriers 116 or functionalized digested decellularized tissue microcarriers 232 can be used for cell transplantation back into the patient from which a tissue sample containing cells expanded to arrive at cultured cells 504 was initially harvested. In stark contrast to microcarriers that are commercially available, which are either non-biodegradable or synthetic and must be separated from the cells for therapeutic applications, one of the advantages of the decellularized mammalian tissue microcarriers 116 and functionalized digested decellularized tissue microcarriers 232 of the present invention over conventional MCs is that the decellularized mammalian tissue microcarriers 116 and functionalized digested decellularized tissue microcarriers 232 are based on the same mammalian tissue of origin as the therapeutic expanded cells 504, thus the decellularized mammalian tissue microcarriers 116 or functionalized digested decellularized tissue microcarriers 232 do not have to be removed from the cells 504 prior to implantation. In other words, the cells 504 expanded on the decellularized mammalian tissue microcarriers 116 or functionalized digested decellularized tissue microcarriers 232 of the present invention can be implanted into the same patient from which the mammalian tissue sample and cells contained therein were initially harvested, without the need to remove the decellularized mammalian tissue microcarriers 116 or functionalized digested decellularized tissue microcarriers 232.

In this approach as shown in FIG. 5, the cells 504, which have been expanded on the decellularized mammalian tissue microcarriers 116 or functionalized digested decellularized tissue microcarriers 232 of the present invention and which are contained in cell culture medium 510 inside a bioreactor or other cell culture vessel 508 can be harvested in step 502. Thereafter, the decellularized mammalian tissue microcarriers 116 or functionalized digested decellularized tissue microcarriers 232 containing the cells 504, along with growth factors 514 (e.g., transforming growth factor beta-1 (TGF-beta1) differentiation factor), can be added to a solution 518 that also contains a gel precursor 516 (e.g., a solution of sodium alginate). Then, the solution 518 can be injected to a site within an area of tissue 524 of a patient where tissue regeneration is desired in step 520, such as at a tissue defect 522. The solution 518 can, in some embodiments, be injected into the tissue defect 522 and fill in the defect 522 in the form of a gel 526 using a minimally-invasive arthroscopic technique.

Next, the solution 518 can be hardened in-situ by visible or ultraviolet radiation or other modes of crosslinking. For example, a cross-linking agent 528 (e.g., a divalent or multivalent ion solution like a calcium chloride solution) can be injected into tissue defect 522 in step 530. For example, when a divalent or multivalent ion solution like a calcium chloride solution is used as the cross-linking agent 528, sodium ions are replaced with calcium ions to initiate cross-linking. After injection and crosslinking, the cells differentiate into the desired cell type based on the specific mammalian tissue harvested and based on the specific growth factors utilized and begin to secrete an extracellular (ECM) matrix. Concurrently, the decellularized mammalian tissue microcarriers 116 or functionalized digested decellularized tissue microcarriers 232 degrade as the cells secrete matrix degrading enzymes.

In another approach (not shown), the cells expanded on the decellularized mammalian tissue microcarriers 116 or functionalized digested decellularized tissue microcarriers 232 can be loaded into a pre-formed, growth factor-loaded scaffold, such as a pre-formed collagen, gelatin, alginate, or chitosan scaffold or a scaffold based on a decellularized matrix, and implanted into a cartilage defect using an invasive surgical procedure.

The present invention may be better understood with reference to the following example.

### Example—Articular Cartilage

In the Example, a method of forming decellularized articular cartilage into a microcarrier for the expansion of cells is described that can be implanted into a cartilage defect in a patient.

### Step 1: Tissue Dissection and Decellularization

Frozen bovine cartilage tissue is obtained from a commercially available source. After thawing, the articular cartilage tissue is dissected into small pieces with a scalpel to generate millimeter scale fragments of articular cartilage approximately 2 millimeters (mm)×2 mm×2 mm. For decellularization, the articular cartilage fragments are incubated in a cell lysis buffer containing 10 millimolar (mM) (hydroxymethyl)aminomethane hydrochloride (Tris-HCl) and 1% polyethylene glycol p-(1,1,3,3-tetramethylbutyl)-phenyl ether (Triton X-100) for 24 hours under ambient conditions (e.g., a temperature of about 23° C. and a relative humidity of about 70%) and stirring. Next, the suspension is sonicated at 55 kHz for 2 hours under ambient conditions. The supernatant is then removed and the tissue fragments are washed thoroughly with phosphate buffer saline (PBS). Then, the tissue fragments are incubated in a nuclease solution consisting of 1 U/mL deoxyribonuclease and 1 U/mL ribonuclease in PBS for 72 hours at 37° C. under stirring to remove all nucleic acids (DNA and RNA). Next, the tissue fragments are washed thoroughly with PBS, freeze-dried and stored at −20° C. The freeze-dried, decellularized articular cartilage is hereafter referred to as DAC.

### Step 2: Tissue Digestion and Functionalization

The freeze-dried decellularized articular cartilage fragments are digested with pepsin in a 0.01 M hydrochloric acid solution at 37° C. using well-established protocols until a clear suspension is obtained. Tissue digestion is complete when the suspension turns from milky or translucent color to a clear suspension. Next, the digestion solution pH is raised to 9.0 using 1 M NaOH to inactivate the pepsin enzyme. After enzyme deactivation, the pH is adjusted to 7.5 using 1 M HCl. Next, methacrylic anhydride is added dropwise to the solution (2.5 mL methacrylic anhydride per gram of digested articular cartilage) under rigorous stirring to functionalize the digested cartilage using well-established methods in the art. The reaction is allowed to proceed for 1 hour. After reaction, the mixture is dialyzed against distilled water under ambient condition to remove the unreacted methacrylic anhydride. The functionalized digested articular cartilage macromer solution is then lyophilized to obtain a white foam, hereafter referred to DDAC, and stored at −20° C.

### Step 3: Micronization

Micronization with Decellularized Articular Cartilage

The steps for micronization of decellularized articular cartilage tissue to generate microbeads or microcarriers are described below. First, the freeze-dried articular cartilage fragments from Step 1 are placed in a mortar (cup-shaped ceramic receptacle) filled with liquid nitrogen and crushed and grinded with a pestle. However, it is to be understood that other micronization methods known in the art can also be used for micronization as long as the method does not require heating the tissue fragments above the physiological temperature of about 37° C. Next, the crushed tissue is passed through a sieve to reduce the particle size to less than 300 micrometers (μm). The average particle size after micronization is approximately 200 μm, although the particle size can range from about 100 μm to 500 μm by changing the parameters of micronization process. The particles generated by crushing and grinding in general have random geometries and are not spherical. The DAC MCs are porous with a honeycomb structure with an average pore size of about 10 μm.

Micronization with Digested Decellularized Articular Cartilage.

The steps for micronization of digested decellularized articular cartilage tissue to generate microbeads are described below. A water phase is prepared by dissolving the DDAC from step 2 in an aqueous solution containing a water soluble emulsifier such as polyvinyl alcohol, polyethylene glycol based emulsifiers, biodegradable poly(ethylene oxide-co-lactide-fumarate) (PLEOF) emulsifiers, and other water soluble emulsifiers as known in the art. Chemical agents such as a crosslinking initiator, crosslinking co-initiator, and a crosslinking agent can also be added to the water phase for crosslinking the DDAC if desired. Next, the water phase is emulsified in an edible oil such as palm oil or any other edible oil known in the art. After emulsification, the DDAC water droplets are allowed to gel to stabilize the droplets in oil and form microbeads (MBs), a type of microcarrier (MC). Next, the MBs are separated from the oil phase by centrifugation or other methods known in the art and washed thoroughly with water to remove unreacted compounds, emulsifier, crosslinking initiator and co-initiator, and crosslinking agent. Next, the DDAC MBs are suspended in water and stored frozen at −20° C. This approach results in porous spherical microbeads as an MC with size varying from 50 micrometers (μm) to 500 μm.

### Step 4: Micronized Decellularized Tissue as Microcarriers for Cell Expansion

The postnatal articular cartilage due to its avascular nature and low metabolism lacks the ability for complete self-repair through native healing mechanisms. The Center for Disease Control and Prevention estimates that nearly 27 million Americans suffer from joint pain and stiffness, loss of function and disability. Current treatment methods rarely restore the full function to the joint. Regenerative strategies using cartilage tissue cells or stem cells harvested from the patient's bone marrow or fat tissue plus growth factors placed in a scaffold has the potential to regenerate the injured articular cartilage. There are many safety and regulatory issues with the expansion of human cells on culture plates or synthetic/non-biologic microcarriers as the stress/shock experienced by the cells in these platforms affects the function and fate of the cells.

A solution to this major challenge in regenerative medicine is to use the DAC or DDAC microcarriers from step 3 above as a platform for expansion of cells from the tissue of origin using the following approach. The steps for expansion of patient's cells on a DAC microcarrier or on a DDAC microcarrier are described below.

First, articular cartilage tissue from a non-load-bearing site on the patient's articular cartilage is harvested. Next, the tissue is treated with articular cartilage matrix-degrading enzymes (ACMD enzymes) to release the cells. Alternatively, cells can be harvested from the patient's bone marrow or fat tissue via a biopsy procedure using well-established protocols. The harvested cells are then washed, sorted, and characterized, after which the desired cells are selected for expansion. After suspending the DAC or DDAC microcarriers/microbeads in culture medium in a stirred-tank cell bioreactor or any other suitable cell culture vessel, the desired cells are added to the cell culture vessel. It should be noted that a non-adherent tissue culture bioreactor material should be used to block cell attachment to the reactor surface and promote cell attachment to the MCs. Next, the suspension is cultured in a tissue culture incubator (37° C., 100% humidity, 5% CO₂) for one day for cell attachment to MCs. Then, the suspension is centrifuged, the supernatant containing the unattached cells is removed, and the pellet containing the cells attached to MCs are re-suspended in the desired medium for cell growth and expansion. During expansion, the cell culture medium is replaced with fresh cell culture medium every few days by centrifugation and re-suspension of the cell-laden MCs to replenish the nutrients in the medium. Once the cells grow and reach confluency on the MCs, the medium is replaced with basal medium containing ACMD enzymes, which are the same enzymes used to release the cells from the natural articular cartilage tissue. After MC digestion, the suspension is centrifuged, the cell culture medium is removed, and the cell pellet is re-suspended and added to a new suspension of DAC or DDAC MCs for further expansion. This process can be repeated many times until the desired fold increase in cell number is reached. Each batch of MCs has the potential to increase the cell number by 1000 fold. Therefore, expansion with one MC change can potentially lead to a 1 million-fold increase in cell number.

### Step 5: Cells Expanded on Decellularized Tissue Microcarriers for Cell Transplantation

As the commercially available MCs are either non-biodegradable or synthetic, the MCs have to be separated from the cells for therapeutic applications. One of the advantages of the DAC or DDAC MCs of the present invention and examples over conventional MCs is that the MCs of the present invention are based on the tissue of origin of the therapeutic cells; thus, the MCs do not have to be removed from the cells prior to transplantation. In other words, the cells expanded on DAC or DDAC MCs can be transplanted into the patient from which the cells attached to the MCs were harvested, without the need to remove the MCs. In this approach, the cells expanded on DAC/DDAC MCs and growth factors can be added to a gel precursor solution, injected to the site of regeneration using a minimally-invasive arthroscopic technique, and hardened in-situ by visible or ultraviolet radiation or other modes of crosslinking.

As an example, the articular cartilage cells expanded on DAC or DDAC MC are mixed with a solution of sodium alginate loaded with transforming growth factor beta-1 (TGF-beta1) differentiation factor and injected into a full-thickness articular cartilage defect. Next, a divalent or multivalent ion solution like a calcium chloride solution is injected into the defect site to replace sodium ions with calcium ions and crosslink. After injection and crosslinking, the cells differentiate into chondrocytes and secrete an articular cartilage extracellular (ECM) matrix. Concurrently, the DAC or DDAC MCs degrade as the cells secrete ACMD enzymes. However, in another approach, it is to be understood that the cells that are expanded on DAC or DDAC MCs can be loaded into a pre-formed, growth factor-loaded scaffold, such as a pre-formed collagen, gelatin, alginate, or chitosan scaffold or a scaffold based on a decellularized matrix, and implanted into a cartilage defect using an invasive surgical procedure.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. A microcarrier for cell culture and expansion comprising decellularized mammalian tissue, wherein the microcarrier has an average particle size ranging from about 10 micrometers to about 600 micrometers.

2. The microcarrier of claim 1, wherein the decellularized mammalian tissue originates from a human donor, a specific human patient, or a non-human mammal.

3. The microcarrier of claim 1, wherein the decellularized mammalian tissue originates from embryonic tissue, neonatal tissue, natal tissue, juvenile tissue, or adult tissue.

4. The microcarrier of claim 1, wherein the decellularized mammalian tissue originates from articular cartilage tissue, bone tissue, heart tissue, liver tissue, skin tissue, or gall bladder tissue.

5. The microcarrier of claim 1, wherein the microcarrier is compatible with cells harvested from mammalian tissue of a same type as the decellularized mammalian tissue.

6. The microcarrier of claim 1, wherein the decellularized mammalian tissue is micronized.

7. The microcarrier of claim 1, wherein the decellularized mammalian tissue is digested and functionalized.

8. The microcarrier of claim 7, wherein the decellularized mammalian tissue is cross-linked.

9. The microcarrier of claim 1, wherein the microcarrier has a honeycomb microstructure.

10. The microcarrier of claim 1, wherein the microcarrier includes pores having an average pore size ranging from about 2.5 micrometers to about 20 micrometers.

11. An injectable or implantable tissue regeneration product comprising:
a microcarrier for cell culture and expansion, wherein the microcarrier includes decellularized mammalian tissue and has an average particle size ranging from about 10 micrometers to about 600 micrometers;
mammalian cells harvested from mammalian tissue of a same type as the decellularized mammalian tissue;
one or more growth factors; and
a gel precursor or a pre-formed scaffold.

12. The injectable or implantable tissue regeneration product of claim 11, wherein the decellularized mammalian tissue originates from a human donor, a specific human patient, or a non-human mammal.

13. The injectable or implantable tissue regeneration product of claim 11, wherein the decellularized mammalian tissue originates from embryonic tissue, neonatal tissue, natal tissue, juvenile tissue, or adult tissue.

14. The injectable or implantable tissue regeneration product of claim 11, wherein the decellularized mammalian tissue originates from articular cartilage tissue, bone tissue, heart tissue, liver tissue, skin tissue, or gall bladder tissue.

15. The injectable or implantable tissue regeneration product of claim 11, wherein the decellularized mammalian tissue is digested and functionalized, further wherein the injectable or implantable tissue regeneration product comprises a cross-linking agent.

16. A method of forming a decellularized mammalian tissue microcarrier for cell culture and expansion, the method comprising:
obtaining a mammalian tissue sample;
decellularizing the mammalian tissue sample; and
micronizing the mammalian tissue sample to form the decellularized mammalian tissue microcarrier, wherein the decellularized mammalian tissue microcarrier has an average particle size ranging from about 10 micrometers to about 600 micrometers.

17. The method of claim 16, further comprising:
digesting the decellularized mammalian tissue sample;
functionalizing the decellularized mammalian tissue sample; and
crosslinking the decellularized mammalian tissue sample.

18. The method of claim 16, wherein the mammalian tissue sample originates from a human donor, a specific human patient, or a non-human mammal.

19. The method of claim 16, wherein the mammalian tissue sample originates from embryonic tissue, neonatal tissue, natal tissue, juvenile tissue, or adult tissue.

20. The method of claim 16, wherein the mammalian tissue sample originates from articular cartilage tissue, bone tissue, heart tissue, liver tissue, skin tissue, or gall bladder tissue.

21. The method of claim 16, wherein the decellularized mammalian tissue microcarrier is compatible with cells harvested from mammalian tissue of a same type as the mammalian tissue sample.

22. The method of claim 16, wherein the decellularized mammalian tissue microcarrier has a honeycomb microstructure.

23. The method of claim 16, wherein the decellularized mammalian tissue microcarrier includes pores having an average pore size ranging from about 2.5 micrometers to about 20 micrometers.

24. The method of claim 16, wherein the decellularized mammalian tissue microcarrier is freeze-dried.

25. A method of treating a mammalian tissue defect, the method comprising:
obtaining a decellularized mammalian tissue microcarrier, wherein the decellularized mammalian tissue microcarrier has an average particle size ranging from about 10 micrometers to about 600 micrometers;
expanding cells harvested from a mammalian tissue sample of a same type as the decellularized mammalian tissue microcarrier on the decellularized mammalian tissue microcarrier inside a cell culture vessel; and
injecting or implanting the decellularized mammalian tissue microcarrier with the expanded cells attached thereto into the mammalian tissue defect.

26. The method of claim 25, wherein the decellularized mammalian tissue microcarrier with the expanded cells attached thereto is injected into the tissue defect in conjunction with one or more growth factors and a gel precursor, further wherein a cross-linking agent is injected into the tissue defect.

27. The method of claim 25, wherein the decellularized mammalian tissue microcarrier with the expanded cells attached thereto is loaded into a pre-formed scaffold containing one or more growth factors, wherein the pre-formed scaffold containing the one or more growth factors and the decellularized mammalian tissue microcarrier with the expanded cells attached thereto is implanted into the tissue defect.

28. The method of claim 25, wherein the decellularized mammalian tissue microcarrier originates from a human donor, a specific human patient, or a non-human mammal.

29. The method of claim 25, wherein the mammalian tissue microcarrier originates from embryonic tissue, neonatal tissue, natal tissue, juvenile tissue, or adult tissue.

30. The method of claim 25, wherein the mammalian tissue microcarrier of decellularized mammalian tissue originates from articular cartilage tissue, bone tissue, heart tissue, liver tissue, skin tissue, or gall bladder tissue.
